(19) United States
(12) Patent Application Publication
(10) Pub. No.: US 2013/0023775 A1
(43) Pub. Date: Jan. 24, 2013

(54) Magnetic Reusable Sensor

(71) Applicants: Marcelo M. Lamego, Coto De Caza, CA (US); Hung Vo, Garden Grove, CA (US); Greg Olsen, Trabuco Canyon, CA (US); Cristiano Dalvi, Mission Viejo, CA (US); Sean Merrit, Lake Forest, CA (US)

(72) Inventors: Marcelo M. Lamego, Coto De Caza, CA (US); Hung Vo, Garden Grove, CA (US); Greg Olsen, Trabuco Canyon, CA (US); Cristiano Dalvi, Mission Viejo, CA (US); Sean Merrit, Lake Forest, CA (US)

(73) Assignee: CERCACOR LABORATORIES, INC., Irvine, CA (US)

(21) Appl. No.: 13/550,289

(22) Filed: Jul. 16, 2012

Related U.S. Application Data
(60) Provisional application No. 61/509,572, filed on Jul. 20, 2011.

(51) Int. Cl. A61B 6/00; A61B 5/024

(52) U.S. Cl. 600/479; 600/476

(57) ABSTRACT

A magnetic reusable sensor is configured to attach to a tissue site so as to illuminate the tissue site with optical radiation and detect the optical radiation after attenuation by pulsatile blood flow within the tissue site. The sensor is configured to communicate with a monitor so as to calculate a physiological parameter corresponding to constituents of the pulsatile blood flow determined by the detected optical radiation. The sensor has a reusable emitter and a detector. A disposable wrap removably secures the emitter and the detector to a tissue site via magnetically enhanced receptacles fixedly mounted on the wrap and magnetically enhanced carriers housing the emitter and the detector.

### CROSS-REFERENCE TO RELATED APPLICATIONS

The present application claims priority benefit under 35 U.S.C. §119(e) to U.S. Provisional Patent Application Serial No. 61/509,572, filed Jul. 20, 2011, titled Magnetic Removable-Pad Sensor; which is hereby incorporated in its entireties by reference herein.

### BACKGROUND

Noninvasive physiological monitoring systems for measuring constituents of circulating blood have advanced from basic pulse oximeters to monitors capable of measuring abnormal and total hemoglobin among other parameters. A basic pulse oximeter capable of measuring blood oxygen saturation typically includes an optical sensor, a monitor for processing sensor signals and displaying results and a cable electrically interconnecting the sensor and the monitor. A pulse oximetry sensor typically has a red wavelength light emitting diode (LED), an infrared (IR) wavelength LED and a photodiode detector. The LEDs and detector are attached to a patient tissue site, such as a finger. The cable transmits drive signals from the monitor to the LEDs, and the LEDs respond to the drive signals to transmit light into the tissue site. The detector generates a photoplethysmograph signal responsive to the emitted light after attenuation by pulsatile blood flow within the tissue site. The cable transmits the detector signal to the monitor, which processes the signal to provide a numerical readout of oxygen saturation (SpO₂) and pulse rate, along with an audible indication of the person's pulse. The photoplethysmograph waveform may also be displayed.

Conventional pulse oximetry assumes that arterial blood is the only pulsatile blood flow in the measurement site. During patient motion, venous blood also moves, which causes errors in conventional pulse oximetry. Advanced pulse oximetry processes the venous blood signal so as to report true arterial oxygen saturation and pulse rate under conditions of patient movement. Advanced pulse oximetry also functions under conditions of low perfusion (small signal amplitude), intense ambient light (artificial or sunlight) and electrosurgical instrument interference, which are scenarios where conventional pulse oximetry tends to fail.

Advanced pulse oximetry is described in at least U.S. Pat. Nos. 6,770,028; 6,658,276; 6,157,850; 6,002,952; 5,769,785 and 5,758,644, which are assigned to Masimo Corporation (“Masimo”) of Irvine, California and are incorporated in their entirety by reference herein. Corresponding low noise optical sensors are disclosed in at least U.S. Pat. Nos. 6,985,764; 6,813,511; 6,792,300; 6,256,523; 6,088,607; 5,782,757 and 5,638,818, which are also assigned to Masimo and are also incorporated by reference herein. Advanced pulse oximetry systems including Masimo SET® low noise optical sensors and read through motion pulse oximetry monitors for measuring SpO₂, pulse rate (PR) and perfusion index (PI) are available from Masimo. Optical sensors include any of Masimo LNOP®, LNCS®, SofTouch™ and Blue™ adhesive or fixed sensors. Pulse oximetry monitors include any of Masimo Rad-8®, Rad-5®, Rad®-5v or SatShare® monitors.

Advanced blood parameter measurement systems are described in at least U.S. Pat. No. 7,647,083, filed Mar. 1, 2006, titled Multiple Wavelength Sensor Equalization; U.S. Pat. No. 7,729,733, filed Mar. 1, 2006, titled Configurable Physiological Measurement System; U.S. Pat. Pub. No. 2006/0211925, filed Mar. 1, 2006, titled Physiological Parameter Confidence Measure and U.S. Pat. Pub. No. 2006/0238358, filed Mar. 1, 2006, titled Noninvasive Multi-Parameter Patient Monitor, all assigned to Cercacor Laboratories, Inc. Irvine, Calif. (“Cercacor”), and all incorporated in their entirety by reference herein. Advanced blood parameter measurement systems include Masimo Rainbow® SET, which provides measurements in addition to SpO₂, such as total hemoglobin (SpHb™), oxygen content (SpOC™), methemoglobin (SpMet®), carboxyhemoglobin (SpCO®) and PVI®. Advanced blood parameter sensors include Masimo Rainbow® adhesive, ReSposable™ and fixed sensors. Advanced blood parameter monitors include Masimo Radical-7™, Rad87™ and Rad57™ monitors, all available from Masimo. Such advanced pulse oximeters, low noise sensors and advanced blood parameter systems have gained rapid acceptance in a wide variety of medical applications, including surgical wards, intensive care and neonatal units, general wards, home care, physical training, and virtually all types of monitoring scenarios.

### SUMMARY

One aspect of a magnetic reusable sensor is a sensor configured to attach to a tissue site so as to illuminate the tissue site with optical radiation and detect the optical radiation after attenuation by pulsatile blood flow within the tissue site, the sensor is configured to communicate with a monitor so as to calculate a physiological parameter corresponding to constituents of the pulsatile blood flow determined by the detected optical radiation. The sensor comprises a reusable optical sensor portion having an emitter and a detector. A disposable wrap portion removably secures the emitter and the detector to a tissue site. The disposable wrap portion has a flexible wrap strip defining an emitter aperture and a detector aperture. An emitter receptacle and a detector receptacle are fixedly mounted to the wrap strip over the emitter aperture and the detector aperture, respectively. The emitter and the detector are mounted to the emitter receptacle and the detector receptacle, respectively, and removably held in place with a plurality of magnets. In this manner, when the wrap strip is attached to a tissue site, the emitter transmits optical radiation through the emitter aperture and the detector receives optical radiation from the emitter through the detector aperture.

Another aspect of a magnetic reusable sensor is a physiological monitoring system having an optical sensor attached to a tissue site, a physiological monitor located distal the tissue site and a sensor cable for providing electrical communications between the optical sensor and the physiological monitor. The optical sensor has an emitter for transmitting optical radiation into a tissue site and a detector for receiving the optical radiation after attenuation by pulsatile blood flow within the tissue site. A current-to-voltage converter is disposed proximate the detector for receiving detector current from the detector and transmitting a corresponding voltage through a sensor cable to a physiological monitor.

A further aspect of a magnetic reusable sensor is a sensor configured to attach to a tissue site so as to illuminate the tissue site with optical radiation and detect the optical radiation after attenuation by pulsatile blood flow within the tissue site. The sensor communicates with a sensor processor so as to calculate a physiological parameter corresponding to constituents of the pulsatile blood flow. The sensor has a fixed sensor portion with emitters and a detector and a removable sensor portion magnetically attachable to and detachable from the fixed sensor portion. The removable sensor portion has pads that receive a tissue site and position the tissue site with respect to the emitters and the detector so as to allow the sensor processor to activate the emitters and receive a corresponding signal from the detector indicative of a physiological characteristic of the tissue site.

In various embodiments, an emitter aperture and a detector aperture are defined by the removable sensor portion. Mounts are disposed on the sensor portions that, in an engaged position, align the removable sensor portion relative to the fixed sensor portion so that the emitter aperture is aligned with the emitters and the detector aperture is aligned with the detector. A connector is disposed on the fixed sensor portion and has a reader conductor that electrically communicates with a reader in a sensor processor. A memory element disposed on the removable sensor portion electrically communicates with the reader conductor when the mounts are in the engaged position. A fixed portion one of the mounts is electrically connected to the reader conductor and a removable portion one of the mounts is electrically connected to the memory element. At least one of the mounts is a magnet and at least one of mounts is a low reluctance, low resistance material. A conductive coil is disposed around at least one of the mounts so as to release the mounts when the coil is electrically activated.

Yet another aspect of a magnetic reusable sensor is a sensor configured to attach to a tissue site so as to illuminate the tissue site with optical radiation and detect the optical radiation after attenuation by pulsatile blood flow within the tissue site, the sensor is configured to communicate with a monitor so as to calculate a physiological parameter corresponding to constituents of the pulsatile blood flow determined by the detected optical radiation. The sensor has a reusable portion with at least one optical element. A disposable portion removably secures the at least one optical element to a tissue site. At least one magnet is disposed on at least one of the reusable portion and the disposable portion so as to releasably join the reusable portion to the disposable portion.

In various embodiments, the disposable portion comprises a wrap strip configured to attach at least one optical element to a fingertip. The disposable portion further comprises an optical element receptacle fixedly connected to the wrap strip and configured to removably join at least one optical element to the wrap strip. The optical element receptacle comprises a first embedded magnet configured to removably secure at least one optical element to the optical element receptacle. An optical element carrier has a second embedded magnet with a polarity opposite that of the first embedded magnet. The optical element carrier has a plug and the optical element receptacle has a socket matching the plug.

An additional aspect of a magnetic reusable sensor is a fixed sensor portion having a plurality of emitters and a detector. A removable sensor portion is magnetically attachable to and detachable from the fixed sensor portion. The removable sensor portion has pads that receive a tissue site and position the tissue site with respect to the emitters and the detector so as to allow a sensor processor in communication with the emitters and the detector to activate the emitters and receive a corresponding signal from the detector indicative of a physiological characteristic of the tissue site.

In various embodiments, the magnetic reusable sensor has an emitter aperture defined by the removable sensor portion, a detector aperture defined by the removable sensor portion and mounts disposed on the sensor portions. The mounts, in an engaged position, align the removable sensor portion relative to the fixed sensor portion so that the emitter aperture is aligned with the emitters and the detector aperture is aligned with the detector. A connector is disposed on the fixed sensor portion. A reader conductor is disposed within the connector so as to electrically communicate with a reader in a sensor processor. A memory element is disposed on the removable sensor portion, which is in electrical communications with the reader conductor when the mounts are in the engaged position.

In further embodiments, a fixed portion one of the mounts is electrically connected to the reader conductor and a removable portion one of the mounts is electrically connected to the memory element. At least one of the mounts is a magnet and at least one of mounts is a low reluctance, low resistance material. A conductive coil is disposed around at least one of the mounts so as to release the mounts when the coil is electrically activated.

A further aspect of a magnetic reusable sensor is forming a wrap strip configured to encircle a fingertip, defining an emitter aperture and a detector aperture in the wrap strip, securing receptacles to the wrap strip positioned over the apertures and removably attaching optical elements to the receptacles. Various embodiments involve mounting optical elements in carriers and embedding magnets in each of the carriers and the receptacles. Other embodiments involve interlacing plug portions of the carriers with receptacle portions of the sockets or separately cabling a first plurality of conductors to an emitter and a detector, embedding an information element in the wrap strip and communicating data from the information element through the embedded magnets to a monitor.

### BRIEF DESCRIPTION OF THE DRAWINGS

FIGS. 1-4 are top attached, top detached, bottom attached and bottom detached perspective views, respectively, of a magnetic reusable sensor having a reusable optics portion and an attachable/detachable disposable wrap portion;

FIGS. 5A-E are top, bottom, edge, side and perspective views, respectively, of a disposable wrap portion of a magnetic reusable sensor;

FIGS. 6A-B are assembled and exploded perspective views, respectively, of an emitter;

FIGS. 7A-E are top, side, end, bottom and perspective views, respectively, of an emitter carrier;

FIGS. 8A-B are assembled and exploded perspective views, respectively, of a detector;

FIGS. 9A-E are top, side, end, bottom and perspective views, respectively, of a detector carrier;

FIGS. 10-11 are perspective views of a junction box (J-Box) having a current-to-voltage (I-V) converter in communications with a corresponding detector;

FIGS. 12-13 are perspective views of a junction box (J-Box) having a cable-splitter and a corresponding detector having an onboard current-to-voltage (I-V) converter;

FIGS. 14A-B are detailed block diagrams of a magnetic reusable sensor and corresponding monitor interface for a detector without an onboard I-V converter (FIG. 14A) and a detector with an onboard I-V converter (FIG. 14B);

FIGS. 15-16 are generalized schematics of sensor detector array channels (FIG. 15) and corresponding monitor front-end channels (FIG. 16);

FIG. 17 is a detailed block diagram of a magnetic reusable sensor;

FIGS. 18A-B are perspective views of a finger clip embodiment of a magnetic reusable sensor;

FIGS. 19A-B are top perspective and exploded top perspective views, respectively, of a magnetic removable-pad assembly;

FIGS. 20A-B are bottom perspective and exploded bottom perspective views, respectively, of a magnetic removable-pad assembly; and

FIG. 21 is an exploded, perspective view of a finger wrap embodiment of a magnetic reusable sensor.

### DETAILED DESCRIPTION OF THE PREFERRED EMBODIMENTS

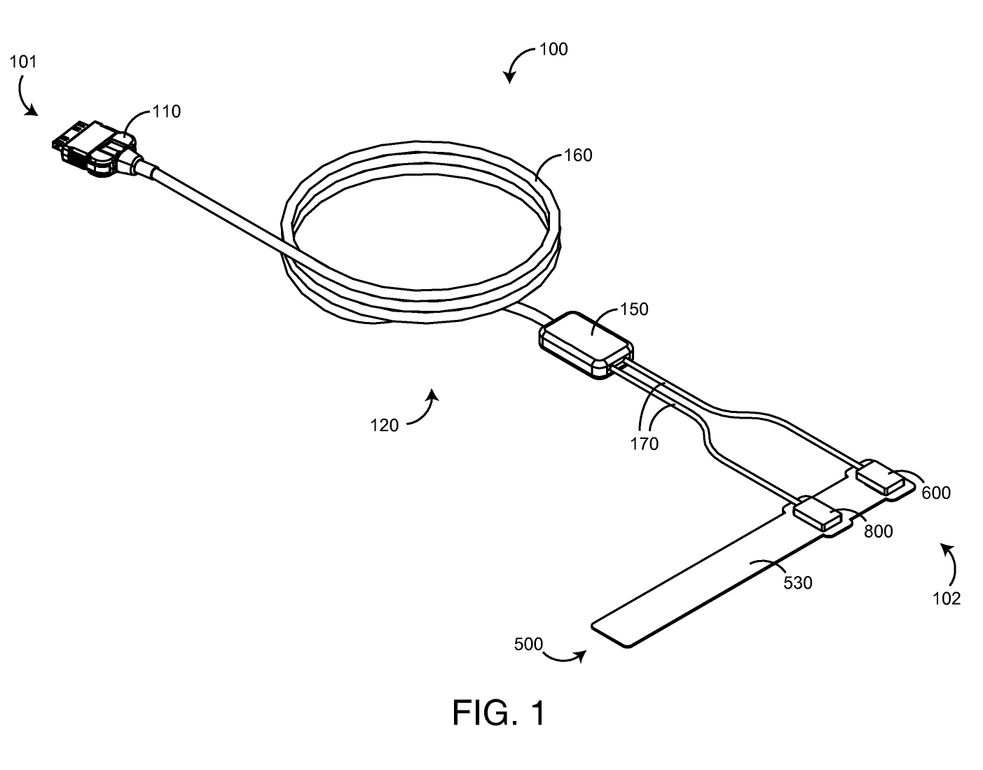

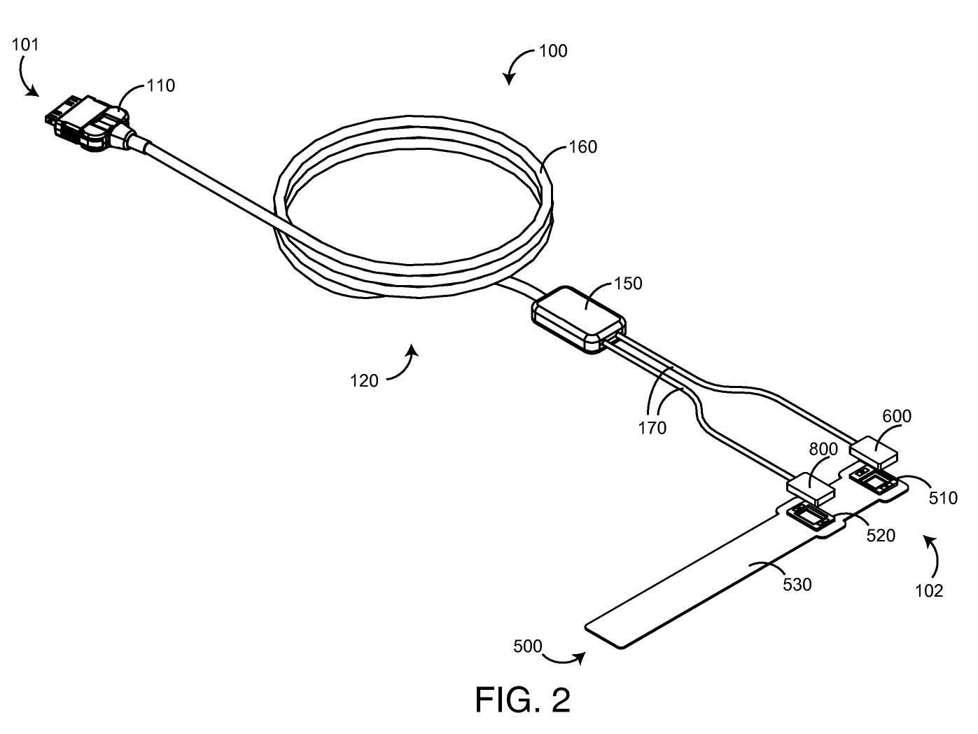

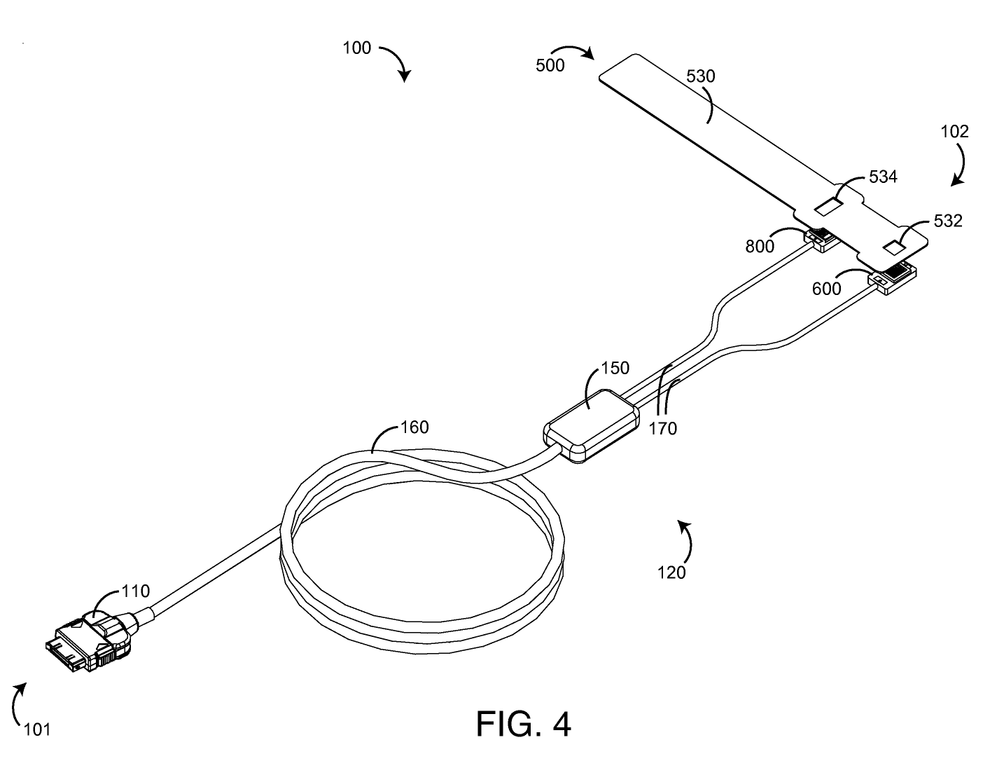

FIGS. 1-4 illustrate a magnetic reusable sensor 100 having a connector end 101, a finger-wrap end 102 and a junction box 150 disposed between the connector end 101 and the finger-wrap end 102. Further, the magnetic reusable sensor 100 has a reusable sensor portion 120 and an disposable wrap portion 500. The reusable sensor portion 120 includes a connector 110, the junction box 150 and a sensor cable 160 disposed between, and in communications with, the connector 110 and the junction box 150. The reusable sensor portion 120 also has an emitter 600, a detector 800 and an optics cable 170 disposed between, and in communications with, the junction box 150 and both the emitter 600 and the detector 800.

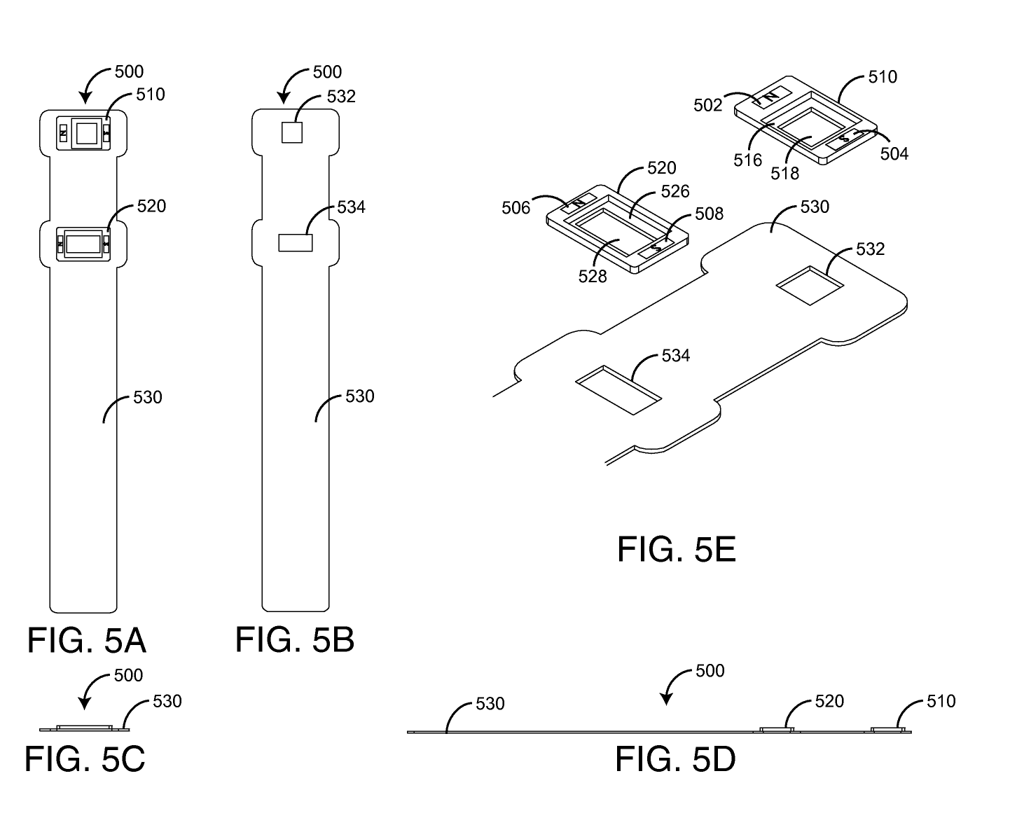

As shown in FIGS. 1-4, the disposable wrap portion 500 has an emitter receptacle 510, a detector receptacle 520 and a wrap strip 530. The wrap strip 530 defines an emitter aperture 532 and a detector aperture 534. The emitter receptacle 510 is fixedly mounted to the wrap strip 530 over the emitter aperture 532. The detector receptacle 520 is also fixedly mounted to the wrap strip 530 over the detector aperture 534. The disposable wrap portion 500 is described further with respect to FIGS. 5A-E, below.

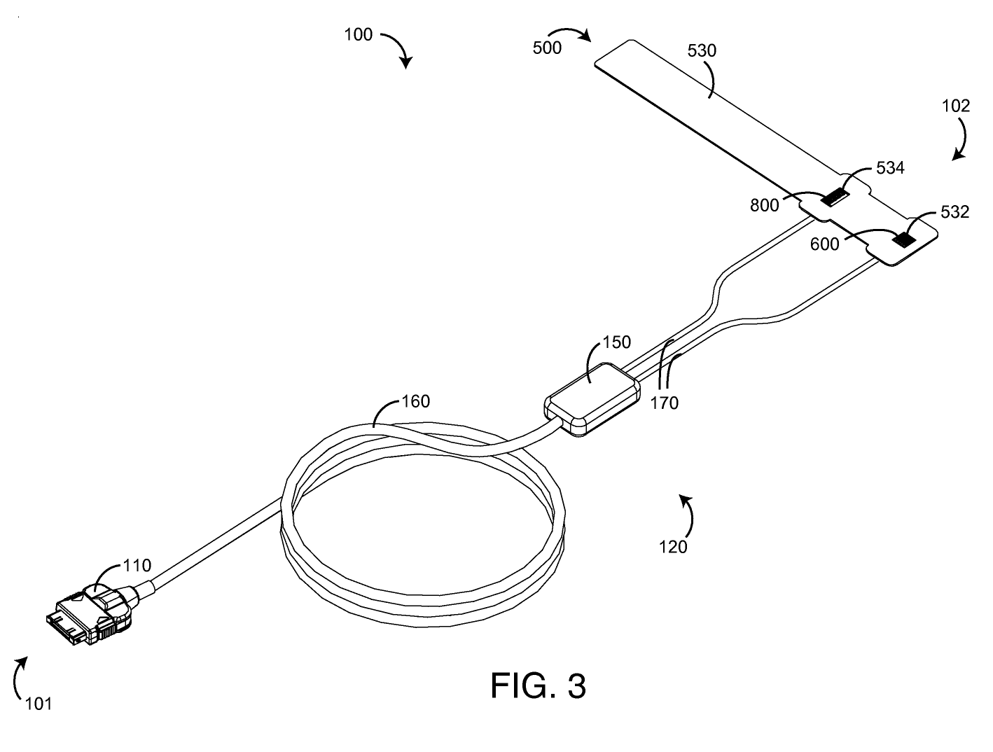

Also shown in FIGS. 1-4, the emitter 600 and the emitter receptacle 510 advantageously incorporate embedded rare-earth magnets so that the emitter 600 removably mounts to the emitter receptacle and, hence, to the finger wrap 500 and so that the emitter optics align with the wrap emitter aperture 532 (FIG. 3). Similarly, the detector 800 and the detector receptacle 520 advantageously incorporate embedded rare-earth magnets so that the detector 800 removably mounts to the finger wrap 500 and the detector optics align with the wrap detector aperture 534 (FIG. 3). In use, the emitter 600 is positioned over the fingernail-side of a finger and the detector 800 is positioned over the fingertip-side of a finger. The emitter 600 and detector 800 are held in place by encircling the wrap strip 530 around the finger and over the emitter 600 and detector 800. An adhesive, Velcro or other fastening mechanism secures the wrap strip 530 in place. In this manner, the emitter 600 transmits optical radiation into the blood perfused tissue beneath the fingernail bed via the wrap emitter aperture 532 and the detector 800 receives optical radiation via the wrap detector aperture 534 after attenuation by pulsatile blood flow within the finger. The emitter 600, emitter receptacle 510, detector 800 and detector receptacle 520 are described further with respect to FIGS. 5-9, below.

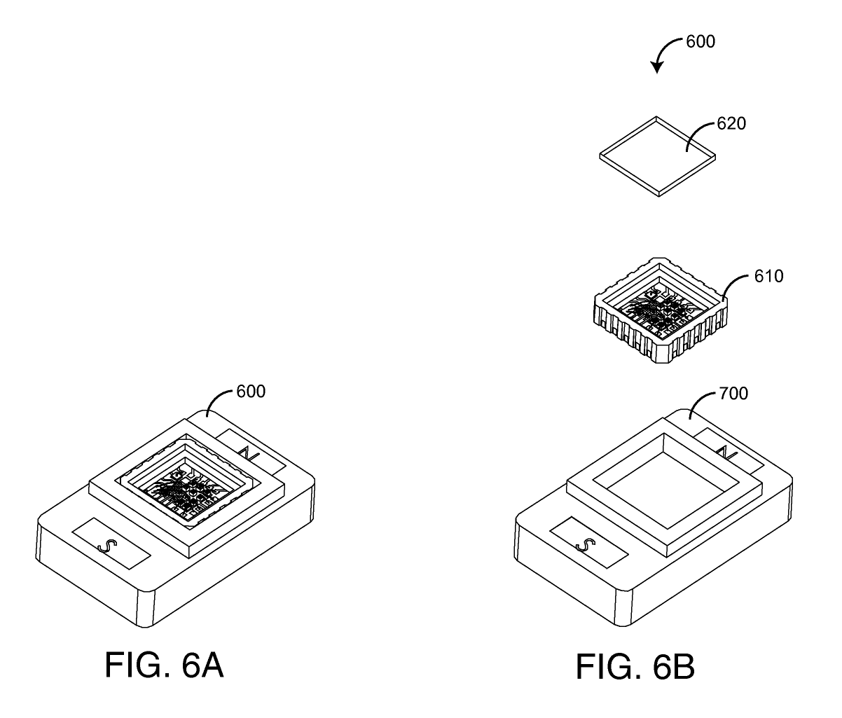

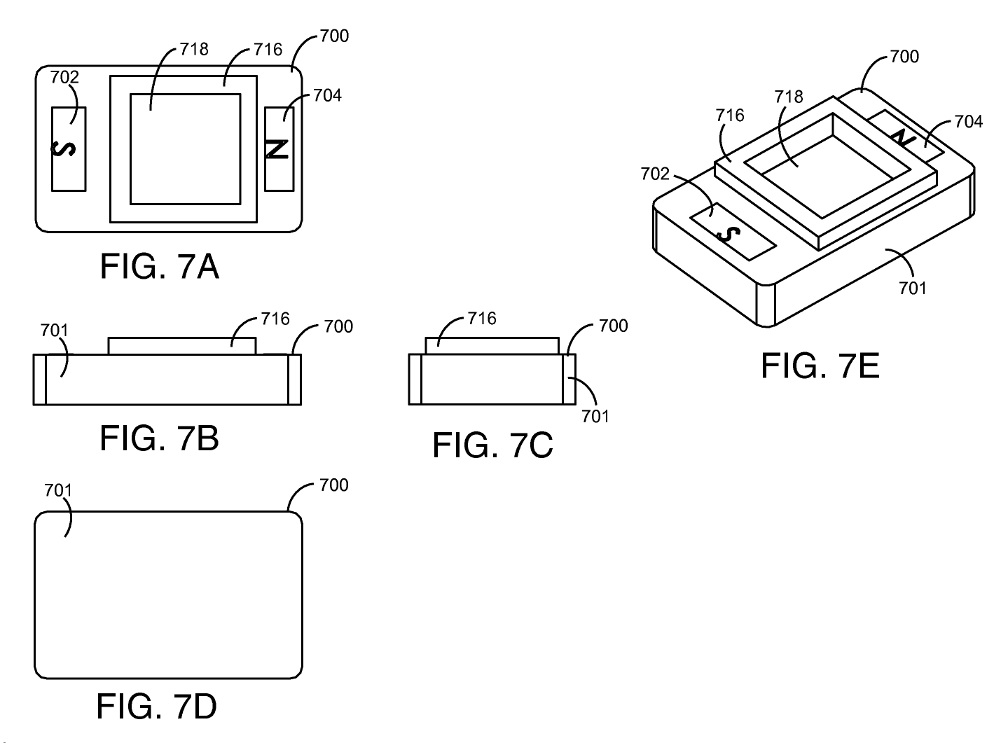

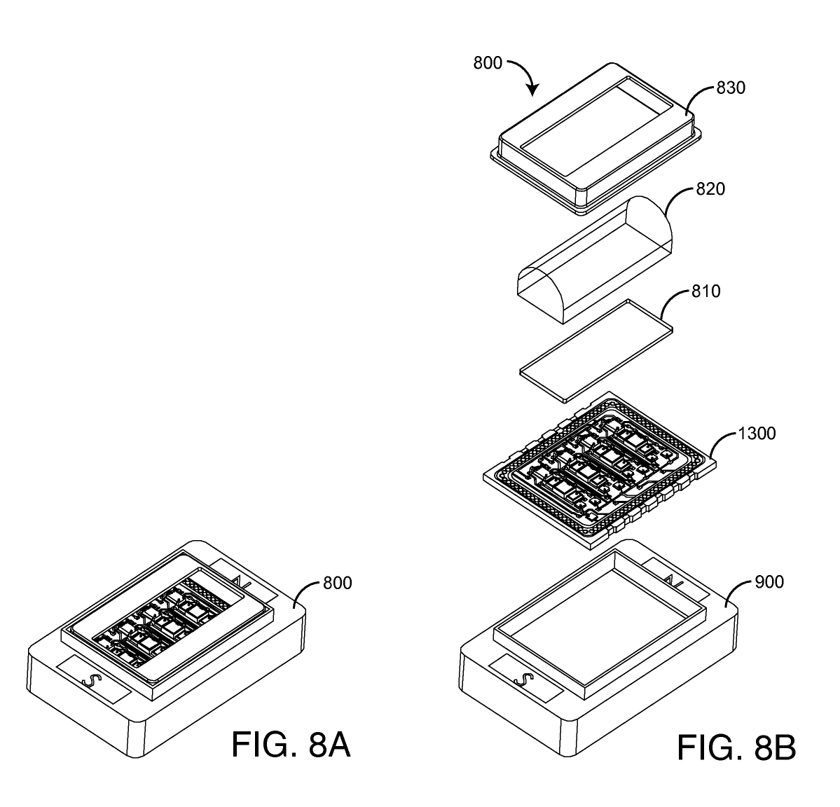

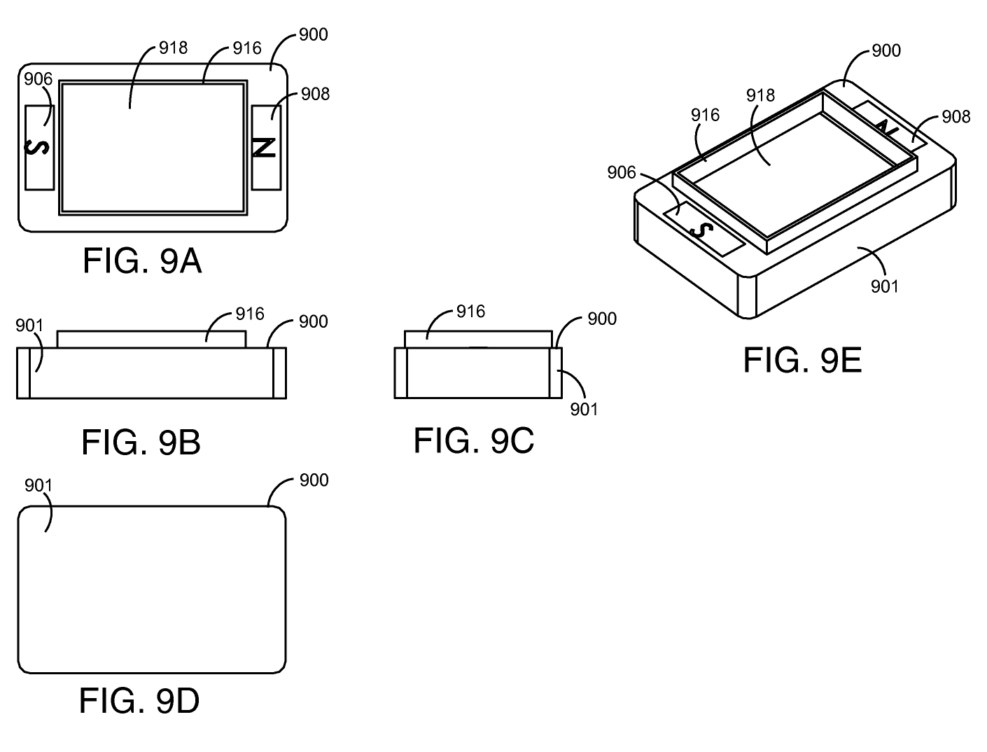

FIGS. 5A-E illustrate a disposable finger wrap 500 having an emitter receptacle 510, a detector receptacle 520 and a wrap strip 530. Advantageously, the emitter receptacle 510 and detector receptacle 520 have embedded magnets with self-aligning, north-south poles so as to removably attach a corresponding emitter 600 (FIGS. 6-7) and detector 800 (FIGS. 8-9), described below. The emitter receptacle 510 is fixedly attached to the wrap strip 530 so that an emitter receptacle aperture 518 aligns with a corresponding wrap emitter aperture 532. The detector receptacle 520 is fixedly attached to the wrap strip 530 so that a detector receptacle aperture 526 aligns with a corresponding wrap detector aperture 534.

As shown in FIG. 5E, an emitter plug 716 (FIGS. 7A-E) is configured to align with and fit within an emitter socket 516. The above alignments are advantageously verified and secured by N and S magnets 502, 504 embedded in the emitter receptacle 510 so as to removably mate with the S and N magnets 702, 704 (FIGS. 7A-E) embedded in the emitter carrier 700 (FIGS. 7A-E). Where “N” and “S” designate a magnet embedded so as to expose its north or south pole, respectively.

Also shown in FIG. 5E, a detector plug 916 (FIGS. 9A-E) is configured to align with and fit within a detector socket 526. The above alignments are advantageously verified and secured by the N and S magnets 506, 508 on the detector receptacle 520 removably mating with the S and N magnets 906, 908 (FIGS. 9A-E) on the detector carrier 900 (FIGS. 9A-E).

FIGS. 6-7 illustrate an emitter 600 for illuminating a tissue site with multiple wavelengths of optical radiation. As shown in FIGS. 6A-B, the emitter 600 has an emitter carrier 700, an emitter circuit 610 and an emitter cover 620. In an embodiment, the emitter circuit 610 comprises a ceramic substrate that mechanically mounts and electrically interconnects a LED emitter array. A ceramic substrate that mounts an LED array is described in U.S. patent application Ser. No. 12/248,841 titled Ceramic Emitter Substrate, filed Oct. 9, 2008, assigned to Masimo Corporation and incorporated in its entirety by reference herein.

As shown in FIGS. 7A-E, the emitter carrier 700 has a base 701 forming an emitter plug 716 surrounding an emitter cavity 718. An emitter “S” magnet 702 and an emitter “N” magnet 704 are embedded at opposite ends of the emitter base 701 so that the south pole of the “S” magnet 702 and the north pole of the “N” magnet 704 are exposed. In an embodiment, the magnets 702, 704 are rare-earth magnets. The emitter circuit 610 is fixedly attached within the emitter cavity 718 so that the LEDs within can radiate outwardly from the cavity 718. The emitter cover 620 is mounted over the emitter circuit 610. In an embodiment, the emitter cover 620 is glass.

FIGS. 8-9 illustrate a detector 800 for receiving multiple wavelength radiation from the emitter 600 (FIGS. 6-7) after attenuation by pulsatile blood flow within a tissue site. As shown in FIGS. 8A-B, the detector 800 has a detector carrier 900, a detector circuit 1300, a detector cover 810, a lens 820 and detector cap 830. In an embodiment, the detector circuit 1300 comprises a ceramic substrate that mechanically mounts and electrically interconnects a detector array.

As shown in FIGS. 9A-E, the detector carrier 900 has a base 901 forming a detector plug 916 surrounding a detector cavity 918. A detector “S” magnet 906 and a detector “N” magnet 908 are embedded at opposite ends of the emitter base 901 so that the south pole of the “S” magnet 906 and the north pole of the “N” magnet 908 are exposed. In an embodiment, the magnets 906, 908 are rare-earth magnets. The detector circuit 1300 is fixedly attached within the detector cavity 918 so that the detectors within can receive optical radiation directed at the cavity 918. The detector cover 810, lens 820 and cap 830 are mounted over the detector circuit 1300.

In an embodiment, an information element is disposed on or within the wrap strip. The wrap strip has conductors in communications between the information element and one or more of the receptacle magnets. Similarly, conductors from the sensor connector 110 (FIG. 1) are in communications with one or more of the carrier magnets so as to allow a monitor to advantageously read the wrap strip information element via the sensor connector 110 (FIG. 1), a carrier magnet, a receptacle magnet and intervening conductors in the sensor cable 160 (FIG. 1) and the wrap strip.

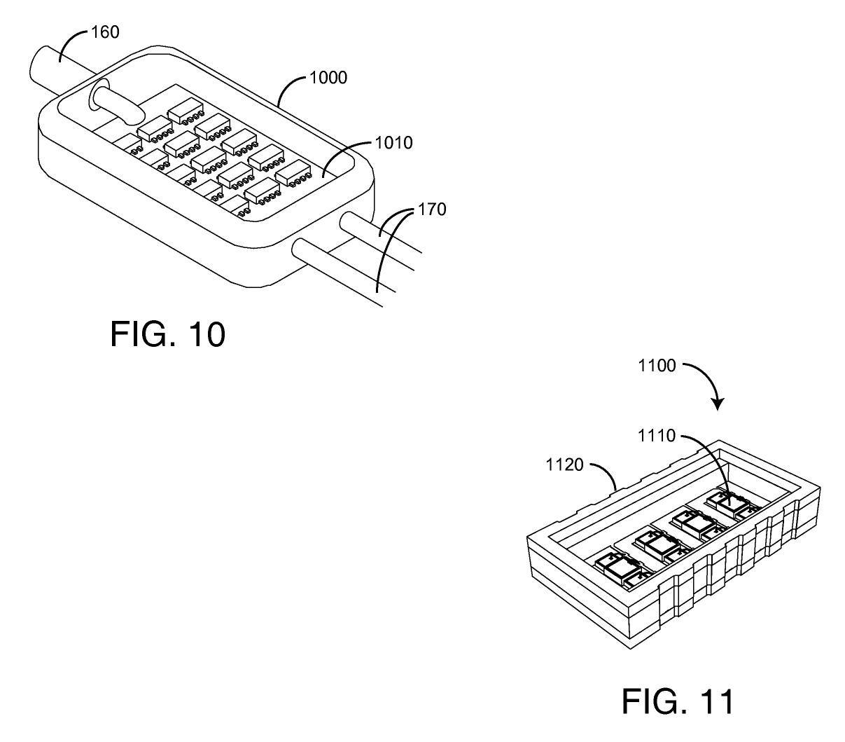

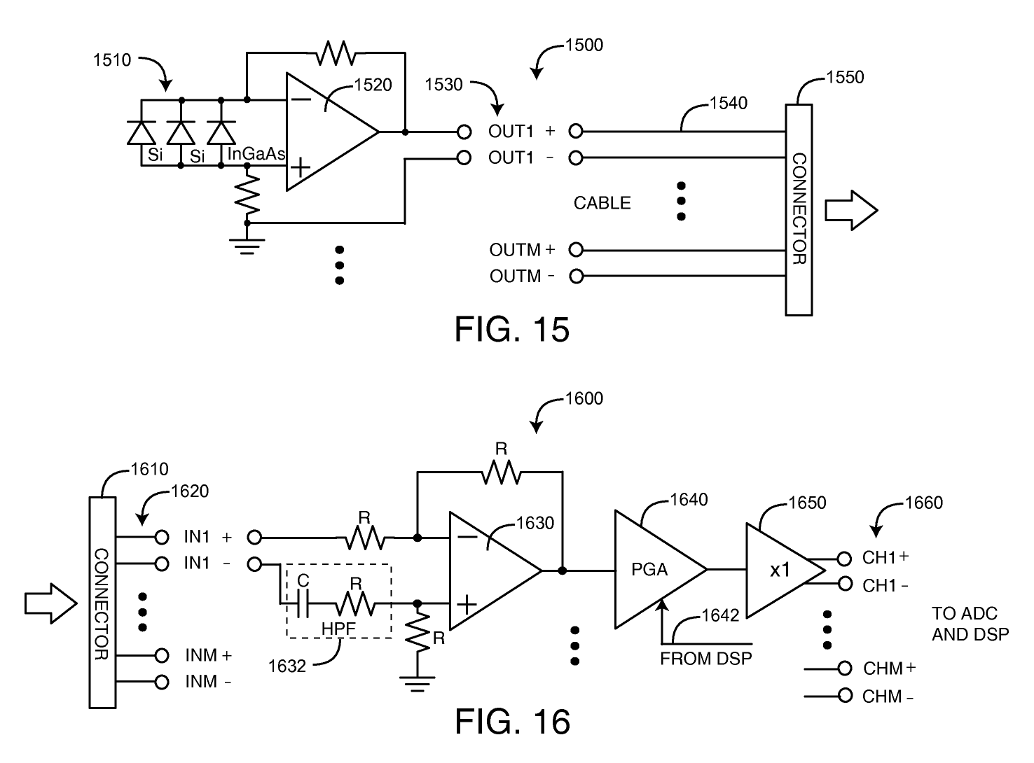

FIGS. 10-11 illustrate a current-to-voltage converter (I-V) junction box 1000 and a corresponding detector 1100. As shown in FIG. 10, a I-V junction box 1000 has a connector-side cable 160 in communications with a monitor connector 110 (FIG. 1); current-to-voltage converter circuitry 1010 mounted within the junction box 1000; and conductors 170 in communications with the emitter 600 (FIG. 1) and the detector 800 (FIG. 1). In an embodiment, the I-V circuitry 1010 comprises transimpedance amplifiers that input current from detector arrays 1110 (FIG. 11) and generate corresponding voltages to a monitor front-end 1600 (FIG. 16) via a connector 110 (FIGS. 1-4). A transimpedance amplifier embodiment is described with respect to FIG. 15, below.

As shown in FIG. 11, the detector 1100 comprises a detector assembly 1110 mounted within a chip carrier 1120, such as a ceramic package. In an embodiment, the detector assembly 1110 comprises four detector arrays, where each array has one InGaAs detector chip and two Si detector chips, as described with respect to FIG. 15, below.

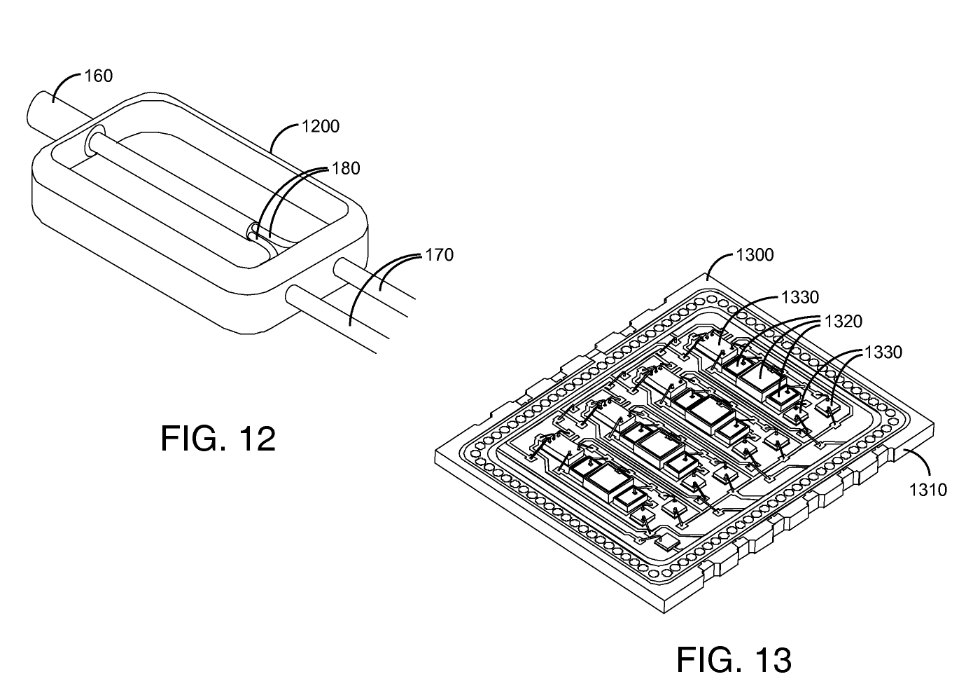

FIGS. 12-13 illustrate a cable-splitter junction box 1200 (FIG. 12) and a corresponding I-V integrated detector 1300 (FIG. 13) having onboard current-to-voltage converters. As shown in FIG. 12, a cable-splitter junction box 1200 has a connector-side cable 160 in communications with a monitor connector 110 (FIGS. 1-4) and split conductors 180 in communications with the emitter 600 (FIGS. 1-4) and the detector 800 (FIGS. 1-4). As shown in FIG. 13, the detector 1100 comprises a chip carrier 1310, a detector assembly 1320 and a current-to-voltage converter assembly 1330. In an embodiment, the current-to-voltage converter assembly 1330 comprises a transimpedance amplifier assembly. In an embodiment, the detector assembly 1320 comprises four detector arrays, where each array has two InGaAs detector chips and one Si detector chip. Detectors and corresponding transimpedance amplifier assemblies are described in further detail with respect to FIG. 15, below.

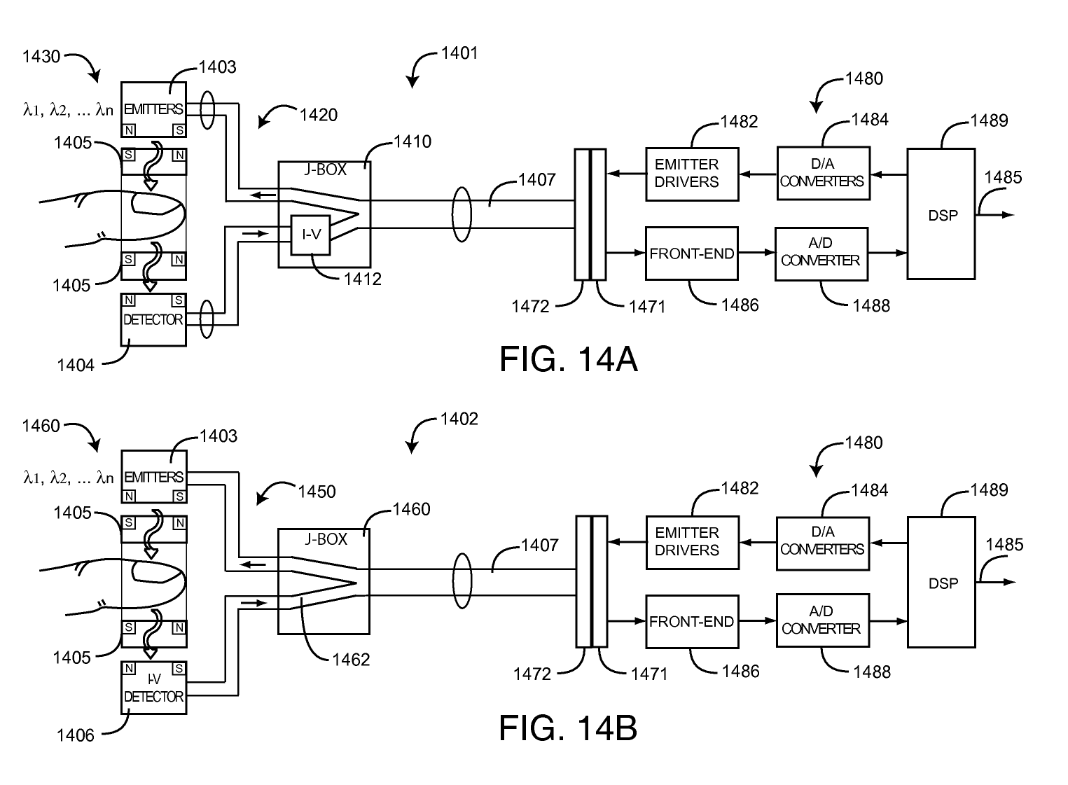

FIGS. 14A-B illustrate magnetic finger-wrap sensor embodiments and corresponding sensor interfaces to a physiological monitor. In either sensor embodiment, 1401 (FIG. 14A), 1402 (FIG. 14B), a physiological monitor 1480 has emitter drivers 1482 that selectively activate sensor emitters 1403 via a sensor cable 1407. In response, the emitters 1403 transmit multiple wavelengths of optical radiation into a tissue site. Detectors 1404, 1406 receive the optical radiation after attenuation by pulsatile blood flow within the tissue site. In an embodiment, the tissue site is a fingertip, and a fingerwrap 1405 advantageously attaches the emitters 1403 and detectors 1404, 1406 to the fingertip via magnetic receptacles 1405, as described with respect to FIGS. 1-4, above. The detectors 1404, 1406 generate a current responsive to the received optical radiation. Current-to-voltage converters (I-V) output a voltage in response to the detector current, which is received by a monitor front-end 1486 via the sensor cable 1407. The detector responsive voltage is processed by the front-end 1486 and digitized by an analog-to-digital converter (ADC) 1488. A digital signal processor (DSP) 1489 controls D/A converters (DACs) 1484, which activate the emitter drivers 1482. The DSP 1489 also inputs detector signals from the ADC 1488 and processes the signals so as to derive physiological parameters accordingly.

As shown in FIG. 14A, a magnetic reusable sensor embodiment 1401 having an associated junction box (J-Box) 1410 with integrated I-V circuitry 1412. The I-V circuitry 1412 interfaces the detectors 1404 output to the monitor front-end 1486, as described above. The I-V circuitry in the J-Box 1410 advantageously allows a relatively stiff shielded cable 1407 to communicate with a relatively flexible, lightly-shielded cable proximate the fingertip. The relative flexibility of the electrical interconnect allows a more robust mechanical connection of the emitters and detectors to the fingertip and greater patient movement and comfort during testing.

FIG. 14B illustrates a magnetic reusable sensor embodiment 1402 having detectors 1406 with integrated I-V circuitry. Accordingly, the associated J-Box 1460 contains only a cable splitter 1462 that separates the emitter 1403 and detector 1406 interconnects. The detector-integrated I-V circuitry advantageously allows a relatively stiff shielded cable 1407 to communicate with a substantially flexible unshielded or lightly shielded cable 1450 proximate the fingertip. The substantial flexibility of this electrical interconnect allows a significantly robust mechanical connection of the emitters and detectors to the fingertip and substantially greater patient movement and comfort during testing. Additional advantages of moving the I-V circuitry closer to the detectors is greater manufacturability, lower cost, more flexible cabling, a higher number of wires per cable, better interference rejection and higher gains in the transimpedance amplifiers.

FIGS. 15-16 illustrate a sensor detector (FIG. 15), which receives optical radiation from multiple wavelength emitters 600 (FIGS. 6A-B) after attenuation by pulsatile blood flow in a tissue site, such as a fingertip, and a corresponding monitor front-end (FIG. 16) that transmits the detected optical radiation to an analog-to-digital converter (ADC) and digital signal processor (DSP) so as to calculate physiological parameters accordingly. As shown in FIG. 15, current generated by a detector array 1510 is converted by a transimpedance amplifier 1520 into a differential voltage output channel 1530 transmitted via cable 1540 to a sensor connector 1550. In an embodiment the detector array 1510 has two silicon (Si) detectors and an indium gallium-arsenide (InGaAs) detector. In an embodiment, there are four detector arrays 1510 corresponding to four sensor output channels 1530.

As shown in FIG. 16, the sensor connector 1550 (FIG. 15) mates with a corresponding monitor connector 1610 so that the sensor output channels 1530 corresponding to monitor input channels 1620. Each monitor input channel 1620 has a differential amplifier 1630 and associated high pass filter 1632, a programmable gain amplifier (PGA) 1642 and a single-end to differential amplifier 1650, which receive, filter and amplifier the transimpedance differential voltage channels 1530 (FIG. 15) into differential ADC input channels 1660. The PGA 1640 variably amplifies the detector signal according to a calibration algorithm that adjusts for patient physiology (e.g. finger size) and, potentially, sensor characteristics (such as pad optical characteristics).

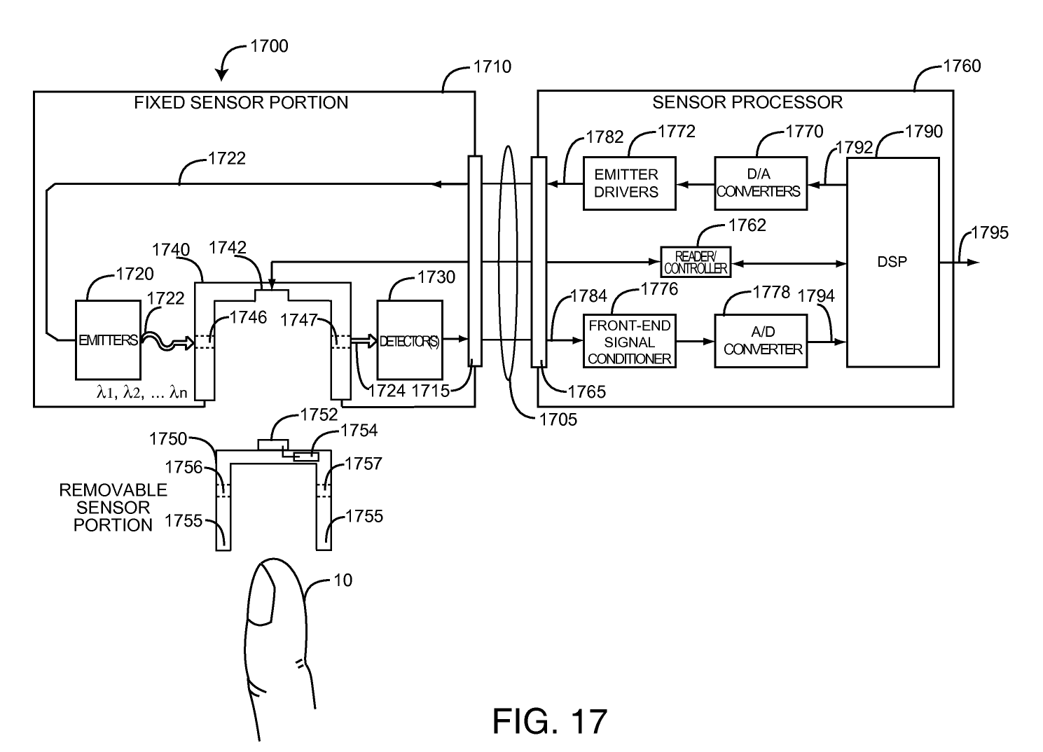

FIG. 17 illustrates a magnetic reusable sensor 1700 embodiment that attaches optical elements 1720, 1730 to a tissue site 10, such as a finger tip, and that mechanically and electrically connects to a physiological monitor 1760. The sensor 1700 has a fixed sensor portion 1710 and a removable sensor portion 1750. The fixed sensor portion 1710 houses optical elements including emitters 1720 and a corresponding detector or detectors 1730. The removable sensor portion 1750 incorporates sensor pads or other surfaces 1755 that come into contact with the tissue site 10. In particular, the fixed sensor portion 1710 has a plurality of emitters 1720 that transmit multiple wavelength optical radiation 1722 and at least one detector 1730 that is responsive to optical radiation 1724 after attenuation by pulsatile blood flow within the tissue site 10. When the removable portion 1750 is mounted to the fixed portion 1740, emitted optical radiation 1722 illuminates the tissue site 10 via a fixed emitter aperture 1746 and a removable emitter aperture 1756. Detected optical radiation 1724 is received via a removable detector aperture 1757 and a fixed detector aperture 1747.

As shown in FIG. 17, the fixed portion 1710 has a receptacle assembly 1740 that accepts the removable portion 1750. The removable sensor portion 1750 attaches to and is held within the fixed portion 1710 via mounts 1742, 1752 that advantageously provide attachment, detachment and electrical communication mechanisms for the fixed and removable portions of the sensor 1700. In particular, the receptacle assembly 1740 has a fixed mount 1742, which mates with a corresponding removable mount 1752, and both mounts 1742, 1752 have relatively low reluctance so that the mounts 1742, 1752 can both securely and magnetically attach the removable portion 1750 to the fixed portion 1740. Further, both mounts 1742, 1752 have a relatively low resistance so as provide electrical communications between a memory element 1754 and a reader 1762. In an embodiment one or both of the mounts 1742, 1752 are permanent magnets that can be physically separated so as to remove and dispose of the removable portion 1750. In an embodiment, one or both of the mounts 1742, 1752 are electromagnets responsive to a controller 1762 so as to release the removable portion 1750 from the mount 1742 for disposal.

Also shown in FIG. 17, the sensor 1710 is configured to communicate with a corresponding sensor processor 1760. In an embodiment, the sensor 1710 has a sensor connector 1715, the processor 1760 has a processor connector 1765 and the sensor 1710 and processor 1760 are in electrical communications via a sensor cable 1705 extending between the connectors 1715, 1765. The processor 1760 has D/A converters 1770 and emitter drivers 1772 that convert digital control signals 1792 from a digital signal processor (DSP) 1790 into analog drive signals 1782 capable of activating the emitters 1720. A front-end 1776, 1778 converts composite analog intensity signal(s) 1784 from the detector(s) 1730 into digital data input 1794 to the DSP 1790. The DSP 1790 may comprise any of a wide variety of data and/or signal processors capable of executing programs for determining physiological parameters from input data. In an embodiment, the sensor processor 1760 may be any of a variety of MX or MS 2000 series OEM circuit boards available from Masimo. In an embodiment, the sensor processor 1760 may be integrated into a wide range of multiparameter and multi-use physiological monitoring devices so as to derive a variety of physiological parameters such as oxygen saturation (SpO₂), carboxyhemoglobin (HbCO), methemoglobin (HbMet), total hemoglobin (Hbt) and oxygen content (OC), to name but a few. Emitters and detectors and corresponding drivers, D/A converters, front-ends and A/D converters are described in U.S. Pat. No. 7,764,982 titled “Multiple Wavelength Sensor Emitters” assigned to Cercacor Laboratories (Cercacor), Irvine, Calif. and incorporated by reference herein.

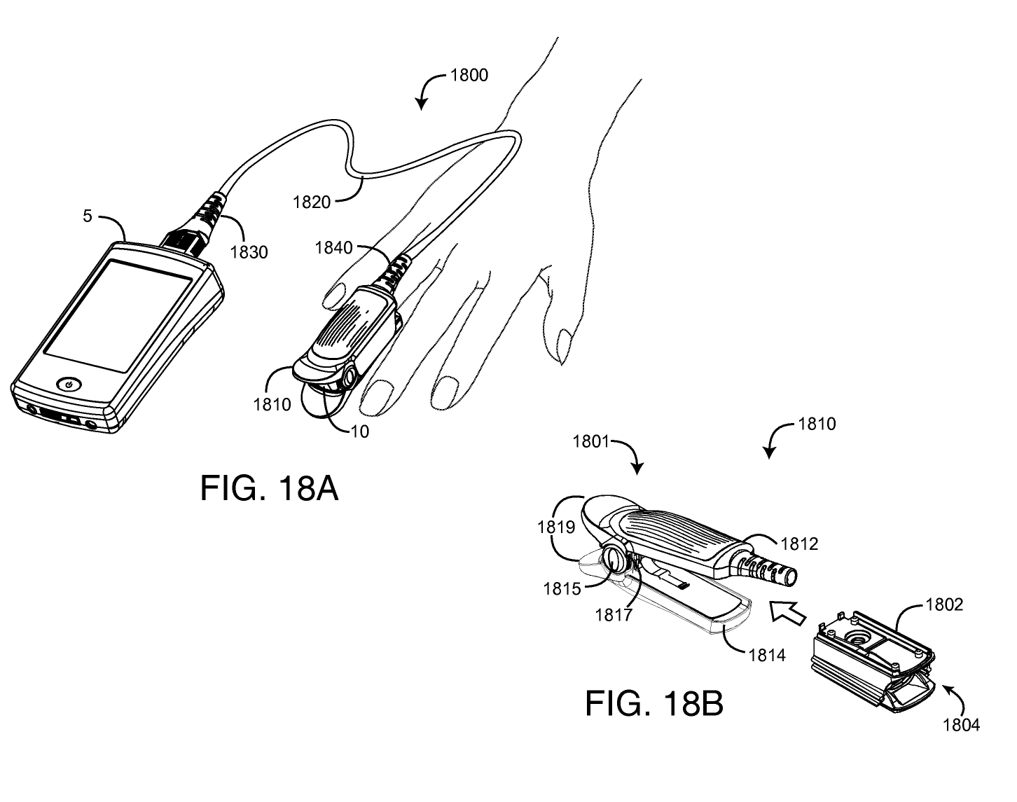

FIGS. 18A-B illustrate a finger clip embodiment 1800 of a magnetic reusable sensor. As shown in FIG. 18A, the finger clip sensor 1800 has a sensor cable 1820 terminating at a monitor connector 1830 at one end and wired to a finger clip 1810 at the opposite end 1840. The finger clip sensor 1800 attaches to a physiological monitor 5 via the monitor connector 1830 inserting into a monitor sensor port (not shown). The monitor may be a handheld device as shown, a standalone instrument or a plug-in to a multi-parameter patient monitor, to name a few. The finger clip sensor 1800 removably attaches to a tissue site 10 via a manual squeeze-and-release action on a finger clip grip 1819. Patient monitors and finger clip sensors are described in U.S. patent application Ser. No. 12/422,915 titled Multi-Stream Sensor for Noninvasive Measurement of Blood Constituents, assigned to Cercacor and incorporated by reference herein.

As shown in FIG. 18B, the finger clip 1810 has a fixed sensor housing 1801 and a removable pad assembly 1802. The sensor housing 1801 includes a finger clip shell 1812, 1814, a pivot pin 1815 and a coiled spring 1817. The pivot pin 1815 rotatably connects a top shell 1812 and a bottom shell 1814 and captures the spring 1817 between the shells 1812, 1814. The spring 1817 urges the top and bottom shells 1812, 1814 together against the tissue site 10. The top shell 1812 houses LED emitters and the bottom shell houses a detector(s). The sensor housing 1801 also positions the emitters and detector relative to the tissue site 10 so as to illuminate the tissue site with multi-wavelength optical radiation and detect that optical radiation after attenuation by pulsatile blood flow within the tissue site. Further, the sensor housing 1801 removably retains the removable pad assembly 1802.

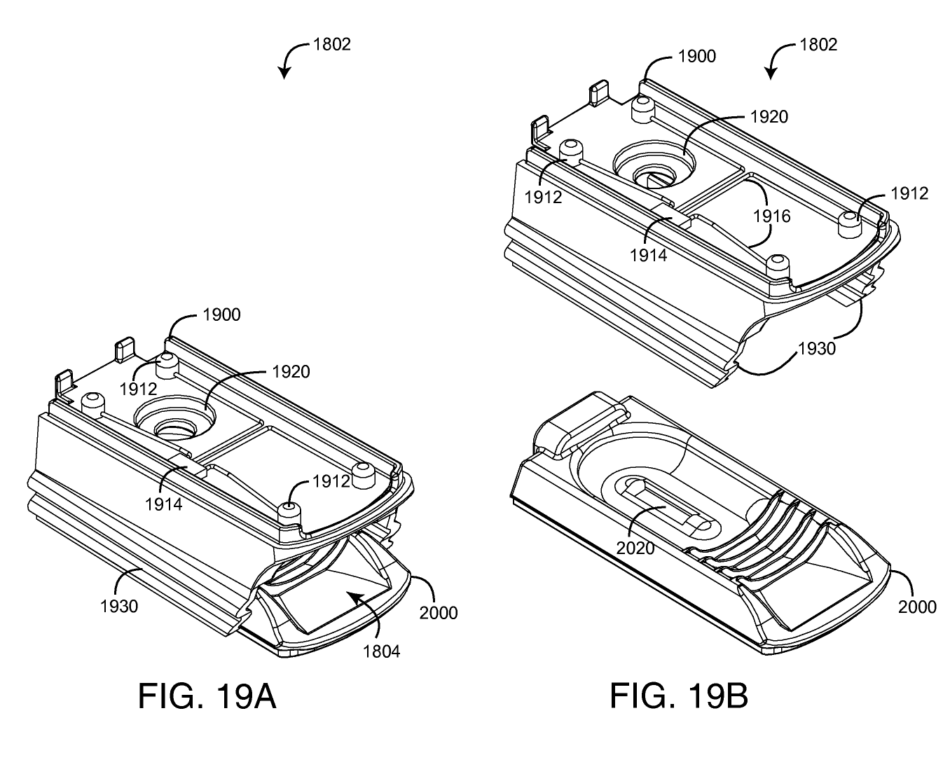

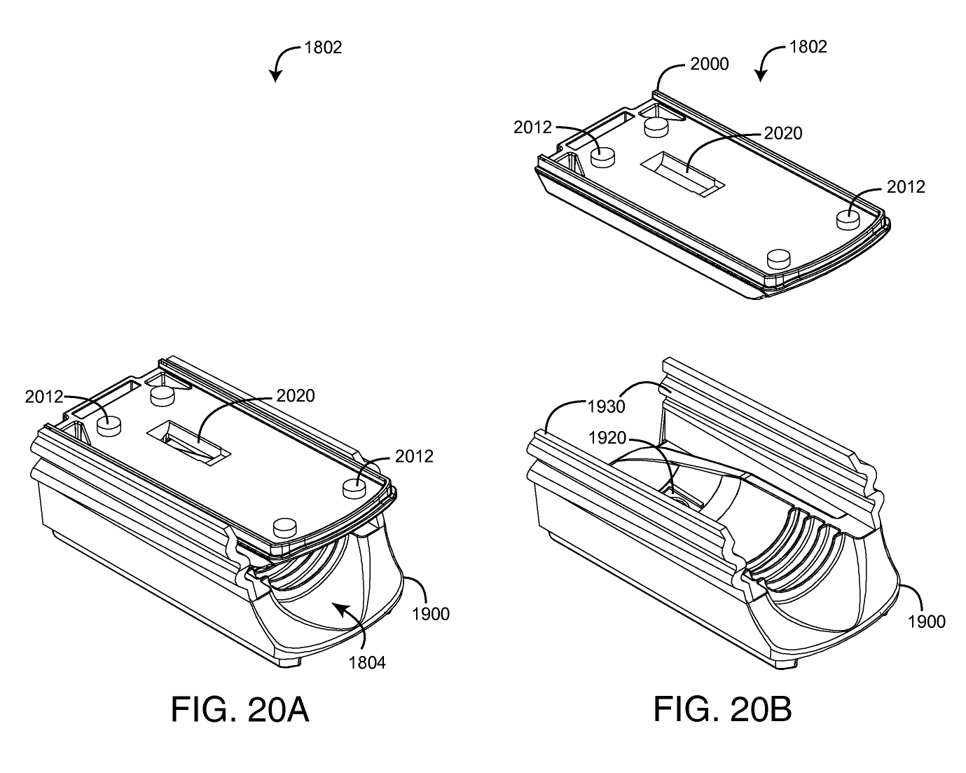

Also shown in FIG. 18B, the pad assembly 1802 receives a tissue site 10, such as a fingertip, via a pad entrance 1804. Inside the pad assembly 1802, the tissue site 10 is cushioned and positioned relative to the sensor housing 1801 and the emitters and detector(s) therein. The pad assembly 1802 is advantageously removably held to the sensor clip via magnet posts 1912, 2012 (FIGS. 19-20), which mate with corresponding metal or magnetic receivers in the housing 1801. The magnets also provide a conductive path so that a memory chip 1914 (FIGS. 19A-B) is in electrical communications with a memory chip reader 1762 (FIG. 17) in the monitor 5 via the sensor cable 1820.

FIGS. 19-20 further illustrate a pad assembly 1802 embodiment. As shown in FIGS. 19A-B, a pad assembly 1802 has a top pad 1900 and a bottom pad 2000. The top pad 1900 has magnetic posts 1912, a memory element 1914, an emitter aperture 1920 and bellows 1930. Conductors 1916 provide communications between the memory element 1914 and the magnetic posts 1912. As shown in FIGS. 20A-B, the bottom pad 2000 has magnetic posts 2012 and a detector aperture 2020. The magnet posts 1912, 2012 are configured to magnetically attach to and electrically connect with corresponding post mounts (not show) in the sensor housing 1801. Bellows 1930 maintain a shield to ambient light while providing for different vertical spacings. The memory element 1914 communicates with a monitor 5 reader so as to provide the monitor with data regarding the pad assembly 1802. In various embodiments, the memory provides manufacturer identification numbers (IDs), optical specifications, test results and usage data, to name a few. IDs can prevent the use of counterfeit, expired or incompatible pad assemblies. Usage data maintains a count of the number of monitor ejections and re-insertions of the pad assembly 1802 (FIG. 18B) relative to the housing 1801 (FIG. 18B).

In an embodiment, the removable pad 1802 can be inserted into or removed from the housing 1801 during manufacture, by an installation representative or by an end user, such as a doctor or other care provider. In an advantageous embodiment, housing mounts are electromagnetic so that the monitor can eject the removable pad 1802 by temporarily inducing an opposing magnetic field. A connector that utilizes an electromagnet to assist in connection and disconnection of a receptacle and plug is described in U.S. patent application Ser. No. 12/721,199 titled Magnetic Connector, filed Mar. 10, 2010, assigned to Cercacor and incorporated by reference herein.

In an embodiment, the pad assembly 1802 is configured for a single use for the most sanitary non-invasive spot check monitoring. In an embodiment, the pad assembly 1802 is configured for finger placement prior to inserting the pad assembly 1802 into the sensor housing 1801. In an embodiment, the pad assembly 1802 is designed for specific patient demographic populations such as pediatric, adult, gender or skin coloration, to name a few. In an embodiment, the pad assembly 1802 is designed for the measurement of particular physiological parameters by incorporating specific pad materials, such as silicone, foam, gel, paper and colors so as to enhance the optical properties of the system for the most accurate readings of specific parameters, specific patient populations or specific disorders. In an embodiment, the memory element 314 has information regarding any or all of the above specified characteristics so as to inform a monitor 5 (FIG. 18A) accordingly.

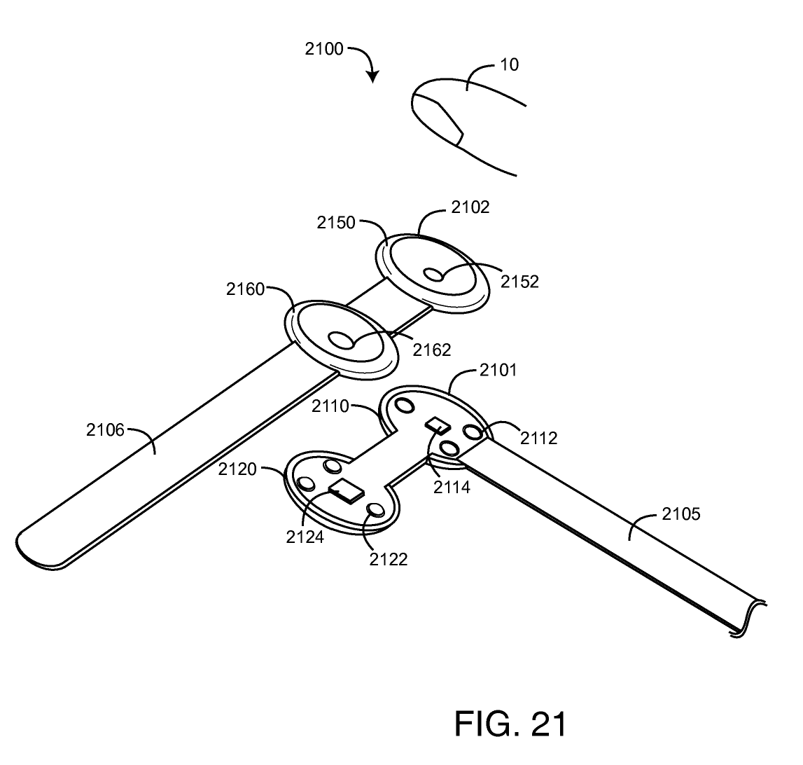

FIG. 21 illustrates a finger wrap 2100 embodiment of a magnetic reusable sensor having a fixed sensor portion 2101 and a removable sensor portion 2102. The fixed sensor portion 2101 has an emitter pod 2110, a detector pod 2120 and a flex cable 2105. The removable sensor portion 2102 has an emitter pad 2150, a detector pad 2160 and a finger-wrap strap 2106. The emitter pod 2110 mounts an emitter 2114 and one or more magnets 2112 that mate with corresponding metal or magnetic receptacles or posts (not shown) in the emitter pad 2150. Similarly, the detector pod 2120 mounts a detector 2124 and one or more magnets 2122 that mate with corresponding metal or magnetic receptacles or posts (not shown) in the detector pad 2160. A flex cable 2105 extends from the emitter pod 2110 and encloses conductors that provide communications between the emitters 2114 and the detector(s) 2124 and a sensor processor 1760 (FIG. 17) or sensor processing portion of a physiological monitor. Further, either the emitter pad 2150 or detector pad 2160 or both may house a memory element 1754 (FIG. 17) that communicates with a corresponding reader 1762 (FIG. 17) in the sensor processor 1760 (FIG. 17) via the flex cable 2105 so as to indicate sensor life, permitted number of uses of the removable portion 2102 or other sensor information as described above.

As shown in FIG. 21, the removable sensor portion 2102 attaches to the fixed sensor portion 2101 so that the emitter pad 2150 attaches to and encloses the emitter pod 2110 and the detector pad 2160 attaches to and encloses the detector pod 2120. A fingernail-side of a fingertip or other tissue site 10 is placed over the emitter pad 2150 so that the emitter 2114 transmits optical radiation into the tissue site 10, such as blood perfused tissue beneath the fingernail bed, via the emitter aperture 2152 of the emitter pad 2150. The strap 2106 is wrapped around the finger so that the detector pad 2160 is placed over the fingertip pad so that the detector 2124 receives optical radiation via the detector aperture 2162 of the detector pad 2160 after attenuation by pulsatile blood flow within the tissue site 10.

A magnetic reusable sensor has been disclosed in detail in connection with various embodiments. These embodiments are disclosed by way of examples only and are not to limit the scope of the claims that follow. One of ordinary skill in art will appreciate many variations and modifications.

What is claimed is:

1. A magnetic reusable sensor is configured to attach to a tissue site so as to illuminate the tissue site with optical radiation and detect the optical radiation after attenuation by pulsatile blood flow within the tissue site, the sensor is configured to communicate with a monitor so as to calculate a physiological parameter corresponding to constituents of the pulsatile blood flow determined by the detected optical radiation, the sensor comprising:
a reusable portion having at least one optical element;
a disposable portion for removably securing the at least one optical element to a tissue site; and
at least one magnet disposed on at least one of the reusable portion and the disposable portion so as to releasably join the reusable portion to the disposable portion.

2. The magnetic reusable sensor according to claim 1 wherein the disposable portion comprises a wrap strip configured to attach the at least one optical element to a fingertip.

3. The magnetic reusable sensor according to claim 2 wherein the disposable portion further comprises an optical element receptacle fixedly connected to the wrap strip and configured to removably join the at least one optical element to the wrap strip.

4. The magnetic reusable sensor according to claim 3 wherein the optical element receptacle comprises a first embedded magnet configured to removably secure the at least one optical element to the optical element receptacle.

5. The magnetic reusable sensor according to claim 4 further comprising an optical element carrier.

6. The magnetic reusable sensor according to claim 5 wherein the optical element carrier has a second embedded magnet having a polarity opposite that of the first embedded magnet.

7. The magnetic reusable sensor according to claim 6 wherein the optical element carrier comprises a plug and the optical element receptacle comprises a socket matching the plug.

8. A magnetic reusable sensor comprising:
a fixed sensor portion having a plurality of emitters and a detector;
a removable sensor portion magnetically attachable to and detachable from the fixed sensor portion; and
the removable sensor portion having pads that receive a tissue site and position the tissue site with respect to the emitters and the detector so as to allow a sensor processor in communication with the emitters and the detector to activate the emitters and receive a corresponding signal from the detector indicative of a physiological characteristic of the tissue site.

9. The magnetic reusable sensor according to claim 8 further comprising:
an emitter aperture defined by the removable sensor portion;
a detector aperture defined by the removable sensor portion;
a plurality of mounts disposed on the sensor portions; and
the mounts, in an engaged position, aligning the removable sensor portion relative to the fixed sensor portion so that the emitter aperture is aligned with the emitters and the detector aperture is aligned with the detector.

10. The magnetic reusable sensor according to claim 9 further comprising:
a connector disposed on the fixed sensor portion;
a reader conductor disposed within the connector so as to electrically communicate with a reader in a sensor processor;
a memory element disposed on the removable sensor portion; and
the memory element in electrical communications with the reader conductor when the mounts are in the engaged position.

11. The magnetic reusable sensor according to claim 10 further comprising:
a fixed portion one of the mounts electrically connected to the reader conductor; and
a removable portion one of the mounts electrically connected to the memory element.

12. The magnetic reusable sensor according to claim 11 wherein at least one of the mounts is a magnet.

13. The magnetic reusable sensor according to claim 12 wherein at least one of mounts is a low reluctance, low resistance material.

14. The magnetic reusable sensor according to claim 13 further comprising a conductive coil disposed around at least one of the mounts so as to release the mounts when the coil is electrically activated.

15. A magnetic reusable sensing method comprising:
forming a wrap strip configured to encircle a fingertip;
defining an emitter aperture and a detector aperture in the wrap strip;
securing receptacles to the wrap strip positioned over the apertures;
removably attaching optical elements to the receptacles.

16. The magnetic reusable sensing method according to 15 further comprising mounting optical elements in carriers.

17. The magnetic reusable sensing method according to 16 further comprising embedding magnets in each of the carriers and the receptacles.

18. The magnetic reusable sensing method according to 17 further comprising interlacing plug portions of the carriers with receptacle portions of the sockets.

19. The magnetic reusable sensing method according to 18 further comprising separately cabling a first plurality of conductors to an emitter and a detector.

20. The magnetic reusable sensing method according to 19 further comprising:
embedding an information element in the wrap strip; and
communicating data from the information element through the embedded magnets to a monitor.
